(19) United States
(12) Patent Application Publication
(10) Pub. No.: US 2015/0012060 A1
(43) Pub. Date: Jan. 8, 2015

(54) REMOTE ELECTRICAL TREATMENT SYSTEM

(71) Applicant: Tung Chi Liu, Kaohsiung City (TW)

(72) Inventor: Tung Chi Liu, Kaohsiung City (TW)

(21) Appl. No.: 13/934,249

(22) Filed: Jul. 3, 2013

(51) Int. Cl. A61N 1/372; A61N 1/36

(52) U.S. Cl. CPC A61N 1/37235; A61N 1/36003
USPC 607/59

(57) ABSTRACT

The remote electrical treatment system contains an authentication server, a prescription server, a backup server, a prescription access device, and an electrical treatment device. A user can requests an electrical treatment prescription anywhere and anytime from the authentication server through the prescription access device, and conducts electrical treatment either when the user is on-lined or off-lined. The present invention therefore frees the user from time and space constraints, provides greater usage flexibility and application support, thereby achieving superior treatment density and enhanced medical quality.

### TECHNICAL FIELD OF THE INVENTION

The present invention is generally related to electrical treatment, and more particular to an electrical treatment system providing remote and real-time electrical treatment.

### DESCRIPTION OF THE PRIOR ART

To prevent muscular dystrophy or degradation to muscle strength, most of paralyzed or disabled patients are required to receive long-term and periodical electrical treatment. However, these patients usually suffer movement difficulty and require other people's assistance. To receive electrical treatment lasing only a few tens of minutes, these patients have to spend much more time and effort. Some patients as such simply give up the treatment. In addition, different patients require different prescriptions. Even through their illness may be identical, their required treatment depth could still be different. Conventionally, an electrical treatment device contains microprocessor, electrical treatment signal generator, and prescription signal transmitter. This simple hardware design and the limited prescriptions are no longer capable of fulfilling the needs of treatment to different ailments.

### SUMMARY OF THE INVENTION

Therefore, a remote electrical treatment system is provided herein. The remote electrical treatment system contains an authentication server, a prescription server, a backup server, a prescription access device, and an electrical treatment device. The authentication server is data-linked to the prescription and backup servers. The prescription server is data-linked to the prescription access device. The prescription access device is data-linked to the electrical treatment device. The backup server stores backup copies of electrical treatment prescriptions of the prescription server. A user requests an electrical treatment prescription from the authentication server and the prescription server through the prescription access device. After being authenticated by the authentication server, an electrical treatment prescription is delivered to the prescription access device, and the user conducts electrical treatment using the electrical treatment device at any location in accordance with the electrical treatment prescription.

The prescription server contains a prescription database and a usage database, both of which are data-linked together. The prescription database stores pieces of information about various electrical treatment prescriptions that can be continuously updated. The usage database keeps track of the usage of various users.

The prescription access device contains a screen, a storage unit, an input unit, an output unit, and an information processing unit connected to the foregoing units. The screen displays electrical treatment prescriptions. The information processing unit connects the authentication server or the prescription server. The storage unit stores electrical treatment prescriptions; the input nit receives input or performs commands. The output unit outputs electrical treatment prescriptions to the electrical treatment device.

The foregoing servers are data-linked through a wired or wireless means.

The electrical treatment device contains electrode pads for exerting electricity on the user's skin.

As described above, the remote electrical treatment system allows a user to receive electrical treatment anywhere and anytime, on-lined or off-lined. The remote electrical treatment system is also capable of providing update and download service to the electrical treatment prescriptions. The inconvenience and insufficiency of conventional electrical treatment is therefore avoided. Through the authentication server's tracking user usage, the treatment density to a user can be truly tracked. In other words, the system is capable of increasing the user willingness to accept electrical treatment and the treatment density, monitoring user's treatment progress. The system is as such capable of enhancing medical efficiency and quality, and reducing waste of social resource.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become apparent to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a functional block diagram of a remote electrical treatment system according to an embodiment of the present invention.

FIG. 2 is a functional block diagram of a prescription access device of the remote electrical treatment system of FIG. 1.

FIG. 3 is a flow diagram showing the operation of the remote electrical treatment system of FIG. 1.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

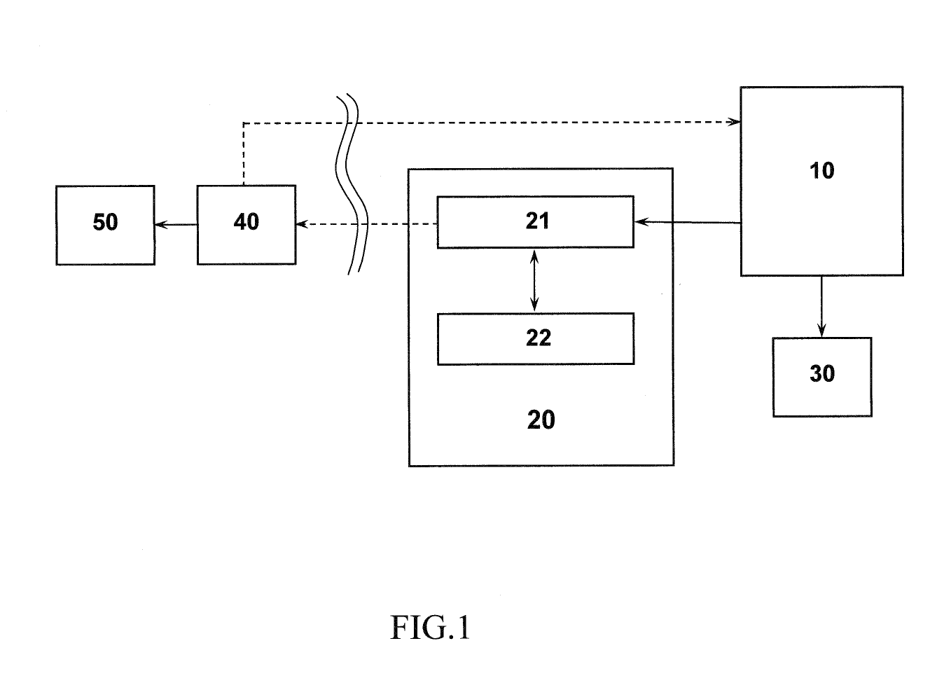

As shown in FIG. 1, a remote electrical treatment system according to an embodiment of the present invention contains an authentication server 10, a prescription server 20, a backup server 30, a prescription access device 40, and an electrical treatment device 50. The authentication server 10 is data-linked to the prescription and backup servers 20 and 30. The prescription server 20 is data-linked to the prescription access device 40. The prescription access device 40 is data-linked to the electrical treatment device 50. The prescription server 20 contains a prescription database 21 and a usage database 22, both of which are data-linked together. The prescription database 21 stores pieces of information about various electrical treatment prescriptions, such as the length of treatment time, strength of electricity, mode of treatment, etc. These pieces of information can be continuously updated. The usage database 22 is for keeping track of the usage of various users. The backup server 30 stores backup copies of electrical treatment prescriptions of the prescription server 20. A user can issues a request through a local prescription access device 40 at any location to a remote authentication server 10 for an electrical treatment prescription. The electrical treatment prescription is then delivered to the local prescription access device 40 from a remote prescription server 20. The user then can conduct electrical treatment using the electrical treatment device 50 in accordance with the delivered electrical treatment prescription.

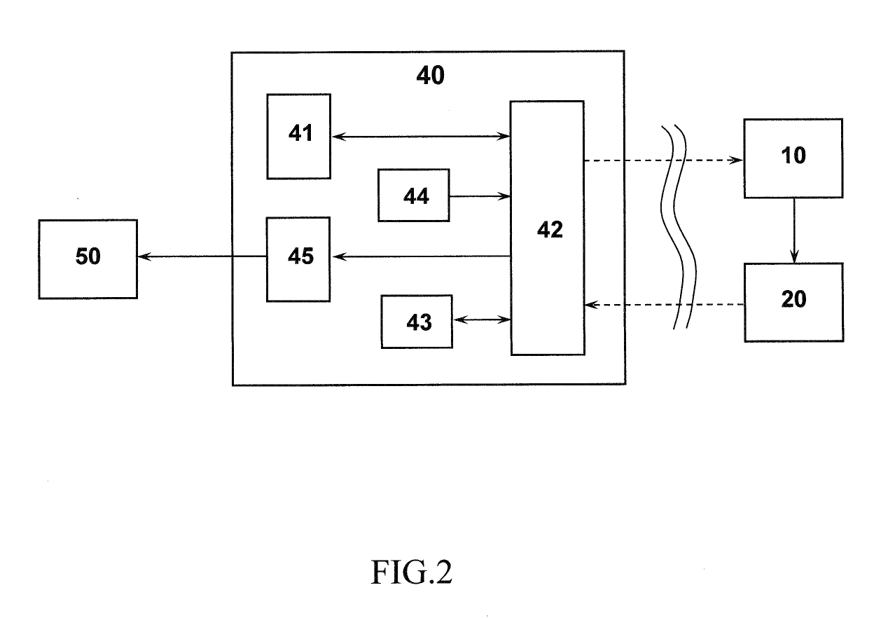

FIG. 2 is a functional block diagram of the prescription access device 40. As illustrated, the prescription access device 40 contains a screen 41, a storage unit 43, an input unit 44, an output unit 45, and an information processing unit 42 linked to all foregoing components. A user can first select the required treatment condition using the screen 41 and the input unit 44 on a local prescription access device 40. The information processing unit 42 then issues a request to a remote authentication server 10 for an electrical treatment prescription through a wired or wireless communication means. After the user is authenticated by the remote authentication server 10, the electrical treatment prescription is then delivered to the local prescription access device 40 from a remote prescription server 20. The electrical treatment prescription is stored in the storage unit 43 for conducting electrical treatment even when the prescription access device 40 is off-lined. The electrical treatment device 50 contains electrode pads (not shown) for attaching to the user's skin. The prescription access device 40 delivers the electrical treatment prescription to the electrical treatment device 50 through the output unit 45. The electrical treatment device 50 exerts the required electrical signals on the electrode pads in accordance with the electrical treatment prescription.

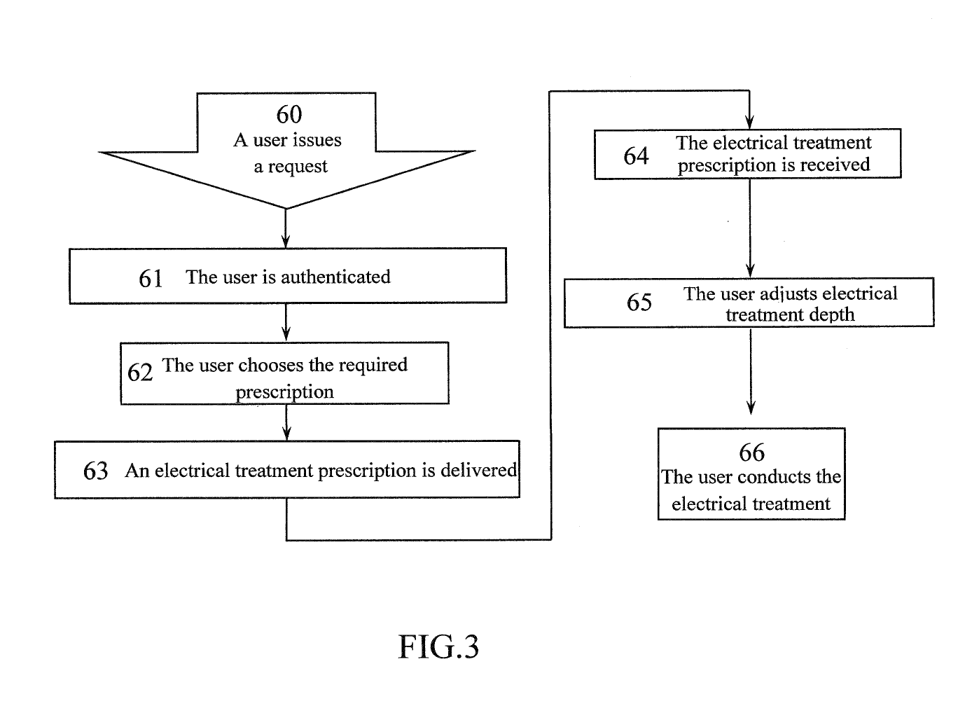

FIG. 3 is a flow diagram showing the operation of the remote electrical treatment system. In step 60, the user issues a request to a remote authentication server 10 for an electrical treatment prescription through a local prescription access device 40. In step 61, the user is authenticated by the remote authentication server 10, and then, in step 62, the remote authentication server 10 provides choices to the user to choose the required prescription. After the user has made the selection, an electrical treatment prescription is delivered to the local prescription access device 40 from a remote prescription server 20 in step 63. The electrical treatment prescription is received and stored by the local prescription access device 40 in step 64. The user then can adjust electrical treatment depth in step 65 and conduct the electrical treatment using the electrical treatment device 50 anywhere or anytime and no matter the user is on-lined or off-lined in step 66. As described above, a user can conveniently receive electrical treatment with very limited time and effort. The user's willingness to take electrical treatment, and the quality and performance of the electrical treatment are all enhanced.

The way that the authentication server 10, the prescription server 20, the backup server 30, and the prescription access device 40 are data-linked can be a wired or wireless means. For wired means, it can be Ethernet, ADSL, VDSL, etc. for connecting to an Internet Service Provider's IP network. For wireless means, it can be GSM, GPRS, PHS, WLAN, WiMax, WiFi, WCDMA, HSDPA, Bluetooth, etc. The prescription access device 40 can be a computer with operating system such Android, iOS, WebOS, BlackBerry Tablet OS, BlueStack, Linux, or Windows. It also should be understandable that the computer can be a desktop computer, a notebook computer, a tablet computer, etc. The screen 41 of the prescription access device 40 can be one of electromagnetic-typed, capacitance-typed, and resistivity-typed touch-sensitive screen. The input unit 44 can provide wired, wireless, or touch means for input. The wired means can be a wired keyboard, a wired mouse, a wired tablet, etc. The wireless means can be a wireless keyboard, a wireless mouse, a wireless tablet, etc. The touch means can be one such as the capacitance-type touch-sensitive device for operating the screen 41.

As described above, the remote electrical treatment system allows a user to receive electrical treatment anywhere and anytime. The user is not required to travel long distance only for treatment of a few tens of minutes. The user is also not required to purchase new hardware for new electrical treatment prescription. Furthermore, the authentication server can be set up by a hospital, and the electrical treatment prescription can be specified by a doctor. The system therefore has a significant level of professionalism and security, and is certainly capable of enhancing medical efficiency and quality, increasing user treatment density, and reducing waste of social resource.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A remote electrical treatment system, comprising an authentication server, a prescription server, a backup server, a prescription access device, and an electrical treatment device; wherein the authentication server is data-linked to the prescription and backup servers; the prescription server is data-linked to the prescription access device; the prescription access device is data-linked to the electrical treatment device; the backup server stores backup copies of electrical treatment prescriptions of the prescription server; a user requests an electrical treatment prescription from the authentication server through the prescription access device; after being authenticated by the authentication server, an electrical treatment prescription is delivered to the prescription access device; and the user conducts electrical treatment using the electrical treatment device at any location in accordance with the electrical treatment prescription.

2. The remote electrical treatment system according to claim 1, wherein the prescription server comprises a prescription database and a usage database, both of which are data-linked together; the prescription database stores pieces of information about various electrical treatment prescriptions that can be continuously updated; and the usage database keeps track of the usage of various users.

3. The remote electrical treatment system according to claim 1, wherein the prescription access device comprises a screen, a storage unit, an input unit, an output unit, and an information processing unit; the screen displays electrical treatment prescriptions; the information processing unit connects the authentication server or the prescription server; the storage unit stores electrical treatment prescriptions; the input nit receives input or performs commands; and the output unit outputs electrical treatment prescriptions to the electrical treatment device.

4. The remote electrical treatment system according to claim 1, wherein the servers are data-linked through a wired or wireless means.

5. The remote electrical treatment system according to claim 1, wherein the electrical treatment device comprises electrode pads for exerting electricity on the user's skin.
